(19) United States
(12) Patent Application Publication
(10) Pub. No.: US 2015/0219621 A1
(43) Pub. Date: Aug. 6, 2015

(54) COVALENTLY BOUND METABOLITES AS BIOMARKERS

(71) Applicant: COUNTERPOINT HEALTH SOLUTIONS, INC., Bedford, MA (US)

(72) Inventor: Wayne Matson, Ayer, MA (US)

(21) Appl. No.: 14/610,779

(22) Filed: Jan. 30, 2015

Related U.S. Application Data
(60) Provisional application No. 61/934,374, filed on Jan. 31, 2014.

(51) Int. Cl. G01N 33/50; G01N 30/64

(52) U.S. Cl. CPC G01N 33/50; G01N 30/64; G01N 2570/00; G01N 2800/304; G01N 2800/302; G01N 2800/28; G01N 2800/2835; G01N 2800/2821; G01N 2800/2814; G01N 2800/042; G01N 2800/32

(57) ABSTRACT

A method for determining the systems biology state of an animal, which comprises determining levels of small molecules covalently bound to macromolecules (CBSM) in samples from that animal, and using said levels to determine the risk, diagnostic state and progression of disease in that animal. A therapy development method for determining the structure of covalently bound molecules and their precursors and modifying the sources and mechanisms causing such binding to reduce disease risk and progression.

### CROSS REFERENCE TO RELATED APPLICATIONS

This Application claims priority from U.S. Provisional Application Ser. No. 61/934,374, filed Jan. 31, 2014.

### BACKGROUND OF THE INVENTION

The present invention relates to the identification of and use of biomarkers for disease and health conditions.

### SUMMARY OF THE INVENTION

This invention is based on the observation that both normal and disease processes result in the covalent binding of small molecules to macromolecules and that these bound forms of small molecules constitute a new class of biomarkers for disease and therapeutic outcome and therapeutic leads that are accessible and measurable with a range of new technologies.

In one aspect of the invention there is provided a method for determining a disease state of an animal much as a human, which comprises determining levels of small molecules covalently bound to macromolecules (CBSM) in samples from that animal, and comparing said levels to standards.

In one embodiment the CBSM small molecules are sourced from gut microbiome derived, derived from metabolic processes, environmental chemical insult or abnormal chemical environment, endogenous or exogenous microorganism, or an interaction of processes between one or more of the above sources.

In another embodiment the macromolecule is selected from the group consisting of DNA, RNA, Protein, a complex carbohydrate and a Glycoprotein.

In yet another embodiment the disease state is selected from the group consisting of disease classification, disease sub categorization, disease progression, development of risk factors predictive of disease, specification of therapy, prediction of therapeutic outcome and development of therapeutic leads.

The present invention also provides a method for therapeutic intervention in disease in an animal much as a human comprising manipulating concentration levels of small molecules covalently hound to macromolecules (CBSM).

In one embodiment the macromolecule is selected from the group consisting of DNA, RNA, Protein, a complex carbohydrate and a Glycoprotein.

In another embodiment the small molecules are sourced from gut microbiome derived, derived from metabolic processes, environmental chemical insult or abnormal chemical environment, endogenous or exogenous microorganism, or an interaction of processes between and among one or more the above sources.

In one embodiment the disease is an affective disease selected from the group consisting of depression, schizophrenia and autism, a degenerative disease selected from the group consisting of Huntington's, Alzheimer's, Parkinson's, Mild Cognitive impairment, ALS, Freidrich Ataxia, cancer, diabetes, and cardiovascular disease, or from in-born errors of metabolism or genetic based disease.

The present invention also provides a method of intervention to prevent or ameliorate disease in an animal much as a human with disease risk which comprises manipulating the animal's small molecules covalently bound to macromolecules (CBSM).

In one embodiment the risk is for an affective disease selected from the group consisting of depression, schizophrenia and autism, a degenerative disease selected from the group consisting of Huntington's, Alzheimer's, Parkinson's, Mild Cognitive impairment, ALS or Freidrich Ataxia, cancer, diabetes, and cardiovascular disease, or from in-born errors of metabolism or genetic based disease.

The invention also provides a method for determining the nature of the source of small molecules covalently bound to macromolecules (CBSM) which comprises creating and analyzing synthetic combinations of small molecules and macromolecules with processes mimicking biochemical processes in an animal much as a human.

The invention further provides a method for modifying gene function in an animal comprising manipulating concentration levels of small molecules covalently bound to macromolecules (CBSM), whereby to increase or decrease expression of a target gene.

In one embodiment the macromolecule is selected from the group consisting of DNA, RNA, a Protein, a complex carbohydrate and a Glycoprotein.

In another embodiment the small molecule is sourced from gut microbiome derived, derived from metabolic processes, environmental chemical insult or abnormal chemical environment, endogenous or exogenous microorganism, or an interaction of processes between and among one or more the above sources.

In yet another embodiment the gene is associated with an affective disease selected from the group consisting of depression, schizophrenia or autism, a degenerative disease selected from the group consisting of Huntington's, Alzheimer's, Parkinson's, Mild Cognitive impairment, ALS, Freidrich Ataxia, cancer, diabetes, and cardiovascular disease, or from in-born errors of metabolism or genetic based.

The present invention also provides a method of therapeutic discovery comprising:

identifying a class of subjects with equivalent genetic risk factors;

identifying sub classes in this class who do and do not develop disease;

identifying differences in covalently bound molecules to DNA, RNA and protein that discriminate the class and subclasses and affect epigenetic differences in the system feedback control; isolating and determining chemical precursor sources and structures of the covalently bound discriminators; and providing or replacing such compounds as are missing or in reduced amount in the disease developing class and/or suppressing such compounds that are elevated or in excess in the disease developing class.

In one embodiment the disease is a neurodegenerative disease selected from the group consisting of Huntington's, Parkinson's, Mild Cognitive Impairment, Amyotrophic Lateral Sclerosis, Freidrich Ataxia, cancer, diabetes and cardiovascular disease, an affective disorder selected from the group consisting of depression, schizophrenia and autism, or from in-born errors of metabolism or genetic based disease.

Finally the invention provides small molecules covalently bound to macromolecules for determination of disease risk, diagnostic status, prediction of disease progression and development of therapy.

### BRIEF DESCRIPTION OF THE DRAWINGS

Further features and advantages of the present invention will be seen from the following detailed description, taken in conjunction with the accompanying drawings, wherein like numerals depict like parts, and wherein;

FIG. 1 schematically illustrates systems biology feedback network determining the state, functionality and risk of diseases for an organism or individual;

FIG. 2 schematically illustrates a preparative flow chart for protein covalently bound small molecule biomarkers and structural identification of same by Mass Spectrometry and location in human plasma;

FIGS. 3A and 3B are LCECA plots of coordinately bound profile and covalently bound profile in plasma (at low Amplification), wherein FIG. 3A shows coordinately bound small molecules extracted from a plasma protein pellet from control subject CC-17, and FIG. 3B shows covalently bound small molecules from the same pellet revealed after digestion with PK;

FIGS. 4A and 4B are LCECA plots of an analytical control showing a PK digest of plasma and a PK digest blank controlling for internal auto cleavage;

FIGS. 5A-5D are LCECA plots showing a comparison of a control (CC-17) and HD (CX53) subject for coordinately bound biomarkers released by acetonitrile (ACN) left hand panel and the PK digest of the pellet from the ACN precipitation;

FIGS. 6A-6B are LCECA plots showing complete digestion of protein pellet from the ACN precipitation of plasma from and HD and Control Subject showing two biomarkers that are descriptive of the Huntington's Disease state;

FIGS. 7A-7B are LCECA plots of fractions from extraction of brain tissue containing a RNA from a K1 CAG 140 HD mouse and a wild type mouse showing eight points of significant difference in the covalently bound small molecules to RNA;

FIG. 8A-8B are plots of nuclear fraction containing DNA from brain tissue of R6/2 mouse and wild type showing eleven points of difference in the covalently bound small molecule adducts; and

FIGS. 9 A-C illustrate the implementation of the flow chart process in FIG. 2 (showing with Indole 3 propionic acid (I3PA) as an example) the preparation of covalently bound small molecule standards, the determination of their binding site and their location in LCECA profiles;

FIG. 9A illustrates creation of a synthetic covalently bound small molecule preparation, and shows schematically a technique for creating standards of covalently bound small molecules through process of creating free radical intermediates of small molecules that can react and covalently bind to macromolecules;

FIG. 9B shows identification of peptide fragment containing the covalently bound kynuric acid fragment of oxidized I3PA and shows that in the synthetic mix of IPA and angiotensin subjected to Fenton reactions that the binding site of its primary oxidation product kynuric acid (KYA) is to a tyrosine moiety and hence detectable at low levels by LCECA; and

FIG. 9C shows Identification of covalently bound I3PA in human subjects using synthetically produced covalently bound material as a standard, and shows a comparison of one channel of an LCECA profile showing that the synthetically produced protein/IPA covalently bound product matches a peak in the array that is lower in control than in HD subjects, and showing in the context of biomarkers of state the ratio of covalently bound IPA (higher in HD) to coordinately bound IPA (lower in HD) is significantly more descriptive as a biomarker than in either compartment alone.

### DETAILED DESCRIPTION

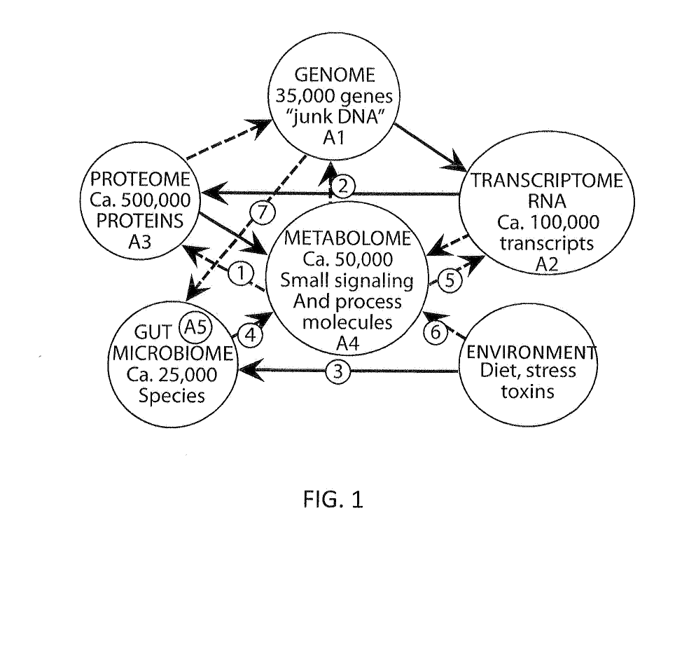

The network of biochemical interactions that define the functional operation of an individual is shown schematically in FIG. 1. Our systems biology concept of disease arises from the implications of this network. Basically disease is not symptoms but rather a failure of control or failure of feedback within this network. Particularly for late onset chronic problems-cardiovascular disease, neurodegenerative diseases, affective disorders, diabetes, chronic fatigue and other triggered immune system problems, symptoms or what we usually call disease arise over time as a result of this failure of control or loss of feedback. Our attempts to define these networks in the context of disease control have focused on multiparameter techniques for finding biomarkers.

Biomarkers, meaning those genes, proteins, RNA transcripts, or small molecules related to disease can generally be classified as: predictive biomarkers, i.e., those that show risk of disease; biomarkers of state, i.e., those that classify disease; biomarkers of progression, i.e.; those that progress with disease; and biomarkers of therapeutic outcome, i.e., biomarkers that change with therapeutic intervention. To these definitions we now add biomarkers that suggest therapeutic intervention strategies.

The search for biomarkers has almost universally been in specific “omic” compartments (A1-A4 in FIG. 1)-looking for genes, gene expression, transcripts, proteins or coordinately bound small molecules. Little has been done to develop techniques and assess the interactions among the “omic” compartments. While not wishing to be bound by theory, we believe and have demonstrated that these interactions have a significant role in providing biomarkers for disease, therapeutic outcome and development of therapies. This invention in part recognizes this lack of “omic” interaction measurements and presents techniques and data for evaluating such interaction.

Small molecule biomarkers are strongly coordinately bound to macro molecules in biological samples. Techniques for assessing small molecule biomarkers (Metabolomics) typically use extraction protocols to remove and concentrate such coordinately bound materials. However, biological/biochemical processes that are either enzyme driven or driven by normal/abnormal free radical production of, for instance, hydroxyl, oxy, or nitro free radical types or simple proximity reactions will cause covalent binding of these closely associated small molecules to macro molecules such as protein, DNA, or RNA. This binding can affect gene expression, the functionality of enzymes and the folding/aggregation of proteins. Since all of the above processes are implicated as risk factors, disease processes and disease progression, the levels and nature of the covalently bound and the distribution of free and coordinately bound small molecules in principal reflects the disease or risk factor processes better than single genes, transcripts, proteins or the totality of coordinately bound and free small molecules.

Referring again to FIG. 1, we have recognized this effect and designed methods to evaluate the feedback linkages 1, 2 and 5 which reflect other feedback processes 3, 4, 6 and 7 and the gut microbiome compartment A5. Several of these methods and processes useful in evaluating are described below in the several non-limiting Examples.

### Example I

Process 1 involves covalently bound small molecule to protein biomarkers for blood-(plasma, leucocytes, platelets, RBC, lysed cells, lysed, whole blood) other bodily fluids and tissue.

In the simplest form protein pellets or other macromolecules (DNA, RNA, complex carbohydrates) derived from preparations using extraction and precipitation of plasma or other tissues for evaluating coordinately bound small molecules were further digested either chemically or enzymatically. The profiles of these preparations were then evaluated with metabolomic techniques such as liquid chromatography with electrochemical detection (LCECA), Mass spectrometry (MS), parallel or series combinations of LCEC/LCMS or nuclear magnetic resonance (NMR).

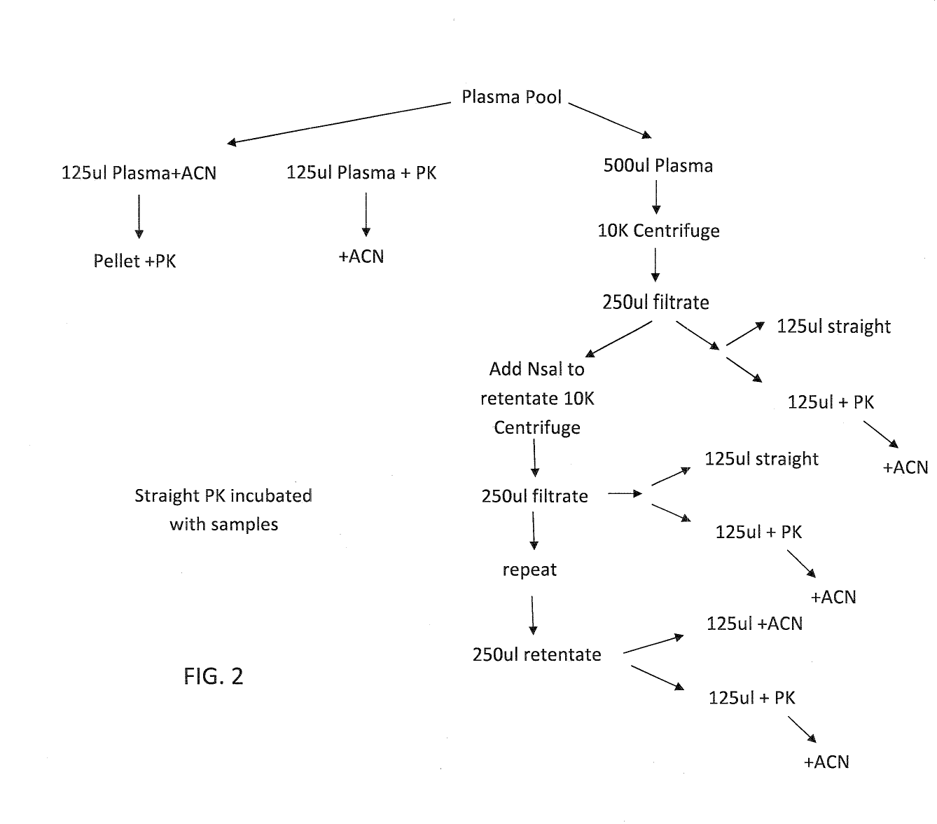

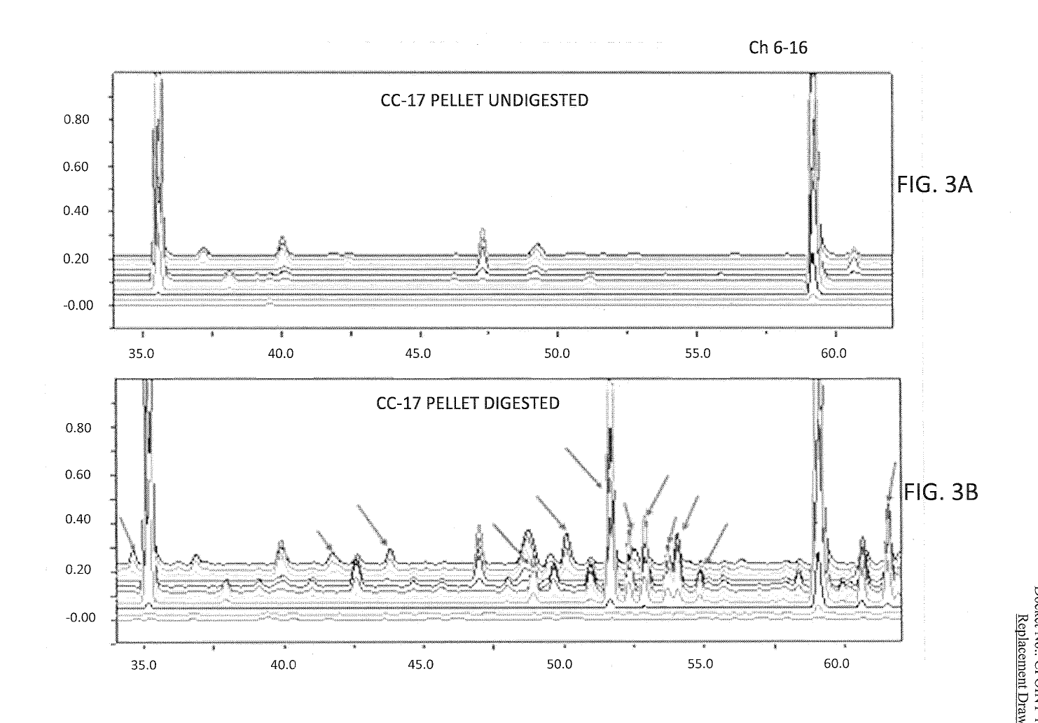

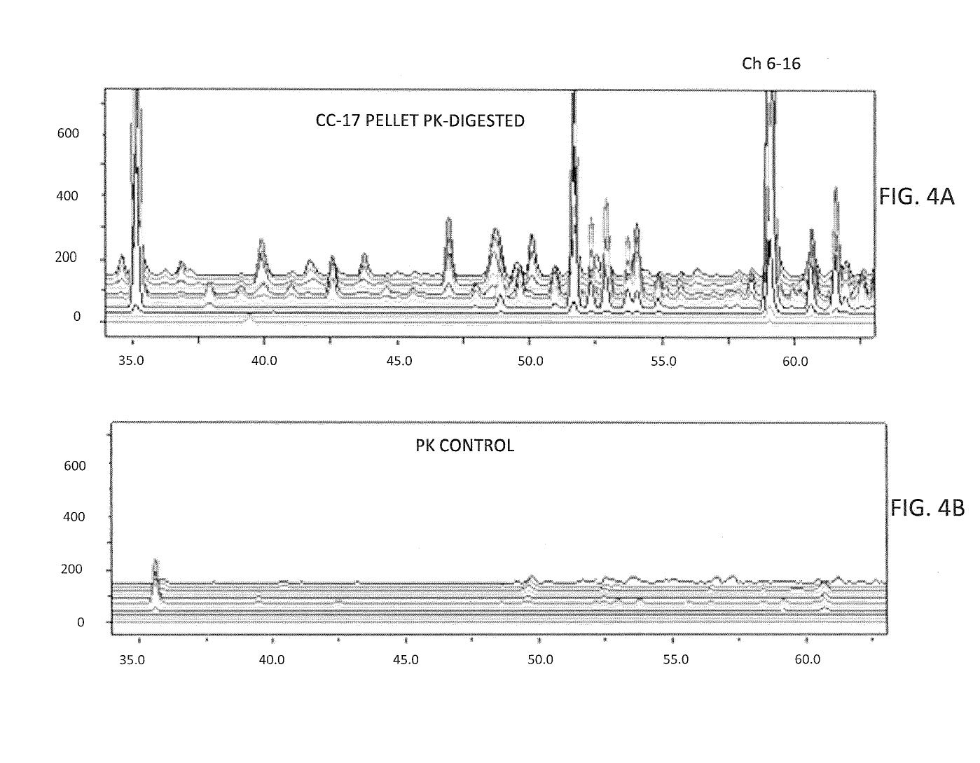

This is shown in the left branch of the sample preparation methodology flow chart in FIG. 2. A typical example from the LCECA profiles of a Control subject plasma for the Acetonitrile extractable fraction of coordinately bound molecules (Top) and the acetonitrile extraction of the protein pellet post digestion with Proteinase K (bottom) is shown in FIGS. 3A-3B. Without derivitization protocols LCECA in the configuration used in this experiment responded only to the amino acids tyrosine tryptophan, and methionine or small dipeptides of these amino acids. The arrows in the bottom figure are compounds from the pellet digestion that are not solely amino acids or small peptides but represent other moieties bound to the amino acid fragment of the digested protein. These bound compounds in terms of biological function will in principle modify the operation of such critical processes as performance of enzymes, protein aggregation, growth or cell death. The subsequent profiles with one technique Liquid Chromatography with electrochemical array (LCECA) detection following the teachings of my earlier U.S. Pat. Nos. 6,194,217 and 6,210,970, showed over 1000 responses which are generally small dipeptides of tyrosine, tryptophan or methionine and small molecules that have been covalently bound to the macromolecules and respond as adduct of the above amino acids. As a control example a digest of human plasma and a PK blank carried through the same process are shown in FIGS. 4A-4B.

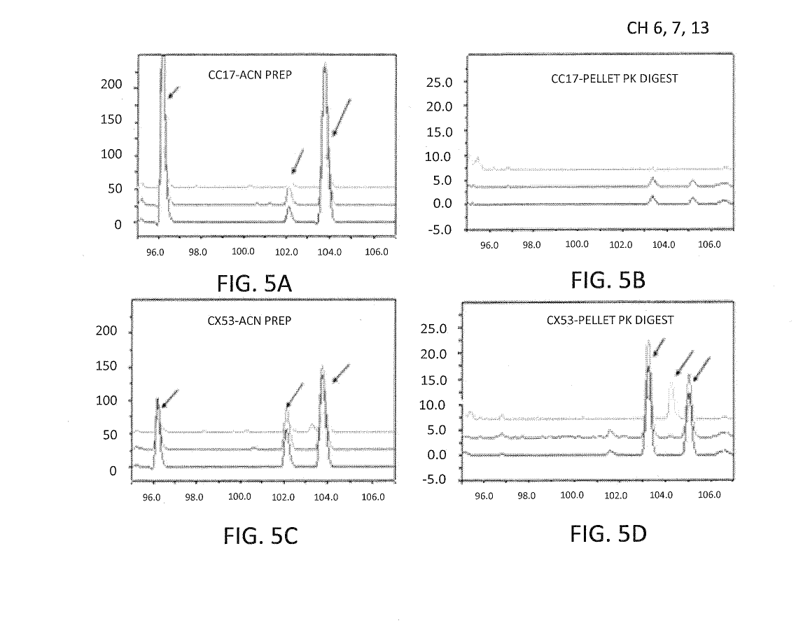

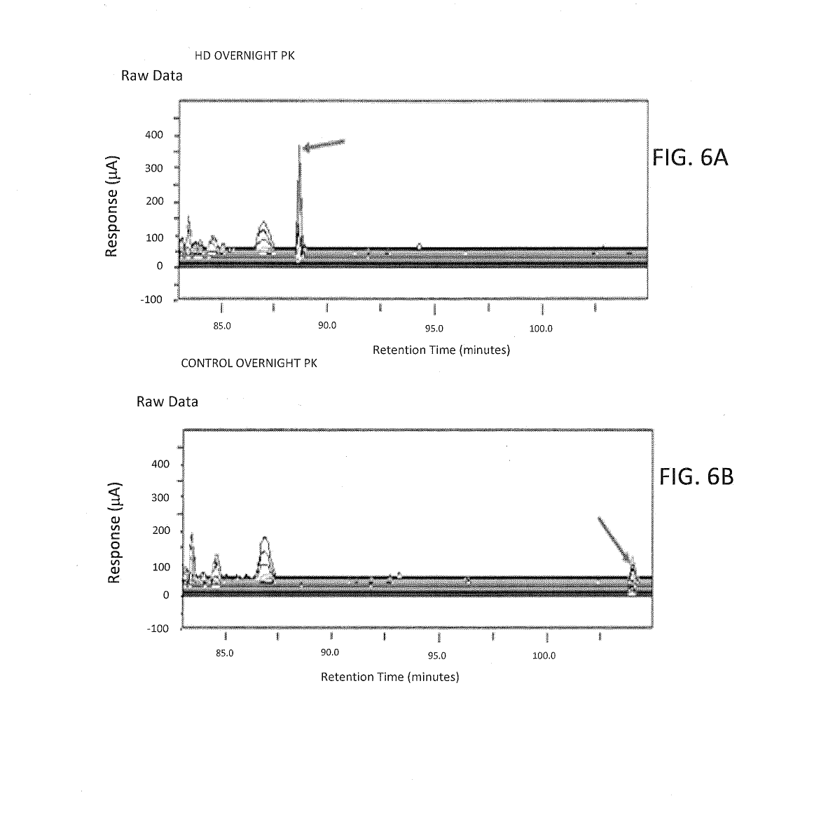

Examples of potential biomarkers of HD vs. controls in this type of preparation are shown in FIGS. 5A-5D and 6A-6B. Notably in the segments of the LCECA profile for the extractable coordinately hound materials shown in FIGS. 5A-5B for a control subject CC17 and HD subject CX53 there are three potential biomarkers of state that are statistically significant. However, in the PK digest profiles of the protein pellet from these subjects shown in FIGS. 5C-5D the biomarkers were of much greater significance. Further in another region of the LCECA profile shown in FIGS. 5A-6B there were two biomarkers of state that alone were completely descriptive and discriminate between disease and control subjects. The process can be extended to fractionation of proteins by size or other means to determine which particular proteins may be most subject to the binding of small molecules and provide both more specific biomarkers of disease or therapeutic outcome or leads to the development of therapies. This was shown in the left side of the sample preparation flow chart in FIG. 2.

Samples were taken through stacked membrane filters in sequence from 1M-300K, 100K-50K-10K molecular weight cut off membranes. The below 10K fraction when processed or analyzed directly reflected the free Metabolome or that which is in equilibrium with coordinately bound fractions in the macro molecules. Sequential macromolecule fractions treated with standard extraction techniques such as precipitation with acetonitrile metha.11ol from which the supernatant was subsequently analyzed via the distribution of coordinately bound molecules as a function of molecular weight.

The analysis of this first set of distributional data provided greater insight into potential biomarkers than the total of all coordinately bound species. For instance the relationship of tryptophan to its primary metabolite kynurinine was partially descriptive of response to antidepressants. However, the relationship of tryptophan to kynurinine in the macromolecule fraction between 300 and 100K was more highly descriptive; the decrease in Indole propionate in AD plasma vs controls was more pronounced in the macromolecule fraction between 100 and 50K, and further pronounced in the ratio of free material to the material bound in the 100 to 50K fraction.

The second set of data was obtained from the macromolecule precipitates in the case of protein precipitates the protein is digested for instance with trypsin (TP) or proteinase k (PK) or beta peptidase or a combination thereof subsequently passing the digest from each fraction through a 10K membrane for PK digests or a 30 K membrane for TP digests and directly analyzing the filtrate.

Additional resolution of other potential markers could be had in the electrochemical array by introducing a boron doped diamond sensor as the last sensor in the series, and further resolution could be had by utilizing LCECA and liquid chromatography with Mass (LCMS) spectrometry in parallel following the teachings of my PCT Application Serial No. PCT/US13/33918, filed Mar. 26, 2013. Essentially any response that does not have the characteristic signature of a peptide in the EC array incorporating a Boron Doped diamond sensor or the extract mass of a peptide in the parallel LCEC/LCMS parallel configuration is a covalently bound small molecule to an amino acid moiety.

### Tissue DNA RNA Preparations

While standard preparative protocols for tissue and DNA/RNA extraction can be used, optimum preparative protocols seek to preserve the macromolecules in the least chemically compromised state. The preparative protocols for tissue involve solubilization of the macromolecules through such processes as grinding of the sample at liquid nitrogen temperatures or using a high speed “tissue mizer” grinder followed by processes such as repetitive freeze thawing in an acceptable matrix such as distilled water or normal saline, or by uses of cycled high pressure disruption again in a suitable matrix.

### Example II

A second approach for clinical samples of whole blood was based on the ability of the LCECA and parallel LCMS platforms to resolve and compare multiple signals quantitatively. A process of isolating DNA from blood by serial filtration through sequentially small pore sizes provided a crude preparation containing DNA that can be sub aliquoted and analyzed with a sequence of extraction preparations for one fraction, and directly lysed with HCl for a second fraction to disrupt the DNA to the base purines and pyrimidines and release covalently bound materials as base adducts. Subsequently the profiles from the two fractions were compared to determine those moieties unique to the DNA.

For DNA, protocols which preserve the histone association with DNA are preferred for initial studies. The histones can be selectively removed by PK digestion and the digests analyzed as above for covalently bound small molecules.

RNA fractions can be evaluated either globally or as isolated using size fractionation protocols to evaluate binding to fractions from tRNA, mRNA exosomes etc. Macromolecule fractions from tissue were evaluated for distribution to various proteins of coordinately and covalently bound compounds from the metabolome are described above. DNA and RNA fractions were evaluated by precipitation/extraction of the coordinately bound metabolome followed by enzymatic disruption as with P 1 endonuclease or P 1 endonuclease followed by AP alkaline phosphatase or digestion with HCl or other weak acid. Purified DNA for instance showed under these protocols the base pairs as 5′ monophosphates (P 1), or the base pairs (P 11AP), or the bases guanine adenine cytidine, thymidine for HCL digests. As we reported in our prior paper, on reviewing entire profiles in our prior paper on 7 methyl guanine. (Anal Biochem. 2013 May 15; 436(2):112-20. doi: 10.1016/j.ab.2013.01.035. Epub 2013 Feb. 12, “A novel method for detecting 7-methyl guanine reveals aberrant methylation levels in Huntington disease”, Thomas B, Matson S, Chopra V, Sun L, Sharma S, Hersch S, Rosas H D, Scherzer C, Ferrante R, Matson W) that other peaks in the response profiles as well as direct modifications of base pairs such as 7 methyl guanine or 8 hydroxy guanine are directly related to other molecules covalently bound to the base pairs or base pair monophosphates or bases. These were made available for assay by the process of dissolution of DNA or RNA and represent species that either inherently respond to the sensors or respond as adducts to the base pairs showing different chromatographic separation.

In our prior paper we also reported on techniques of isolating RNA and DNA from brain tissue with the intent of developing a targeted method for guanine and 7 methyl guanine in DNA and RNA following the hypothesese that changes in the ratio of these would be indicative of epigenetic differences. The whole point of the targeted assay was to get a clean signal for the guanine and 7 methyl guanine which were indeed descriptive of epigenetic changes in the wild type and CAG 140 HD mouse model and in human postmortem HD and control brain. This involved removal of coordinately bound species and substantial manipulation of the LCECA. However when we recognized the potential significance of the “interferences” as covalently bound small molecules we re-analyzed the entire chromatographic output.

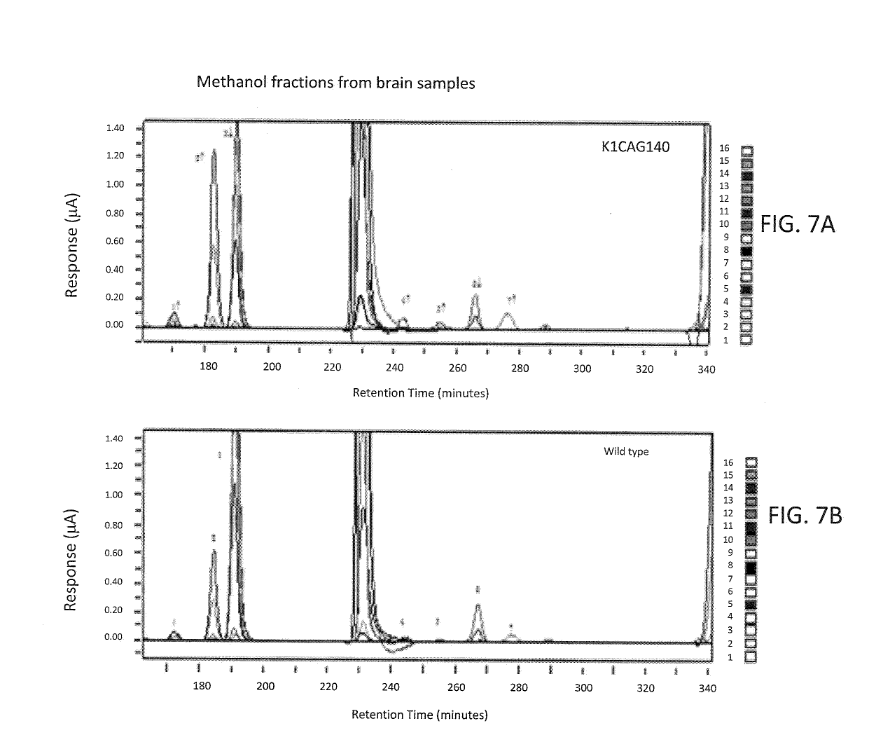

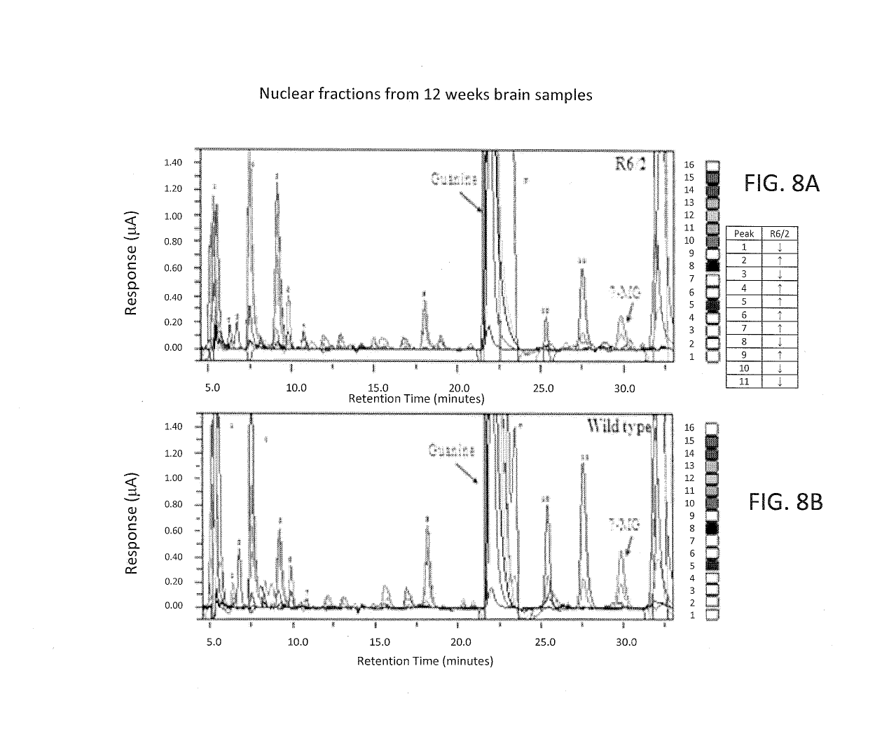

Shown below in FIGS. 7A-7B and 8A-8B are eight other covalently bound species that are significantly different in the KI CAG 140 mouse and wild type mouse RNA (methanol fraction) FIGS. 7A-7B and eleven other covalently bound species that are significantly different in the wild type an R6/2 mouse DNA (nuclear fraction). (FIGS. 8A-8B). These other covalently bound adducts completely discriminate the wild type from the gene modified animals using multivariate PLS-DA with one out testing of the models. It was also observed that the covalently bound species in DNA and RNA differentiated the CAG 140 late onset HD model from the R6/2 early onset model. This suggests that time of phenoconversion in Hf) may be related to specific species binding to DNA and RNA which in turn suggests and approach to therapeutic intervention to delay or prevent onset of symptoms in subjects carrying the Hf) gene.

While not wishing to be bound by theory, it is believed that all of these species are likely involved in the functionality of the DNA and RNA and consequently both reflect and determine the operation of the network shown in FIG. 1. The functionality of this network in turn determines the outcome or disease fate of the individual.

### Example III

Strategies for identifying the source of a covalently bound material:

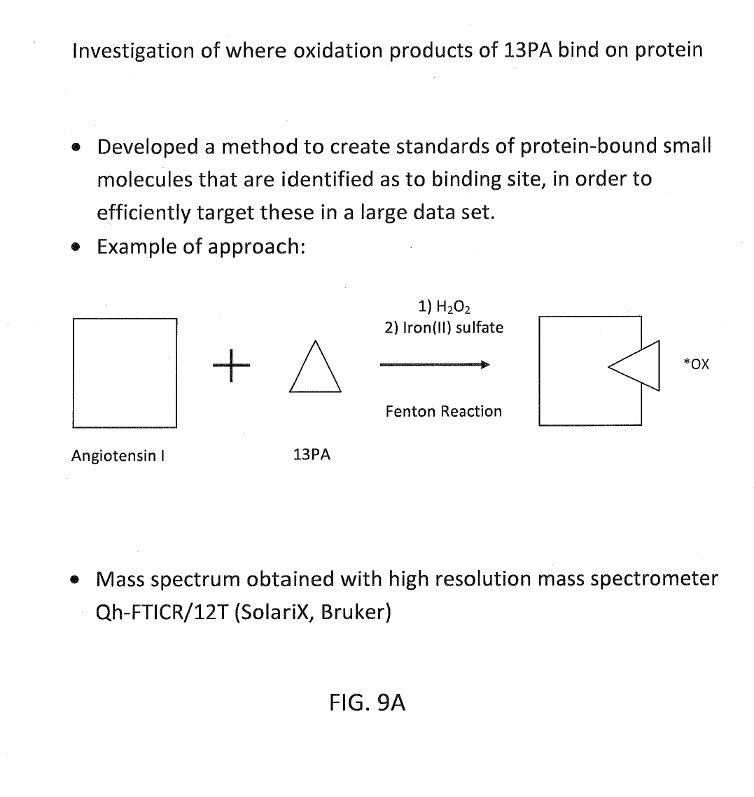

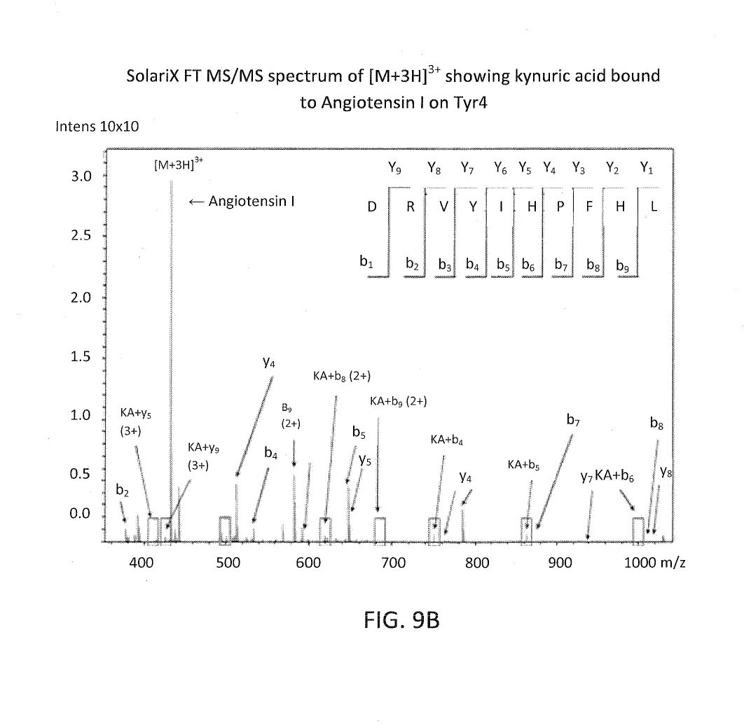

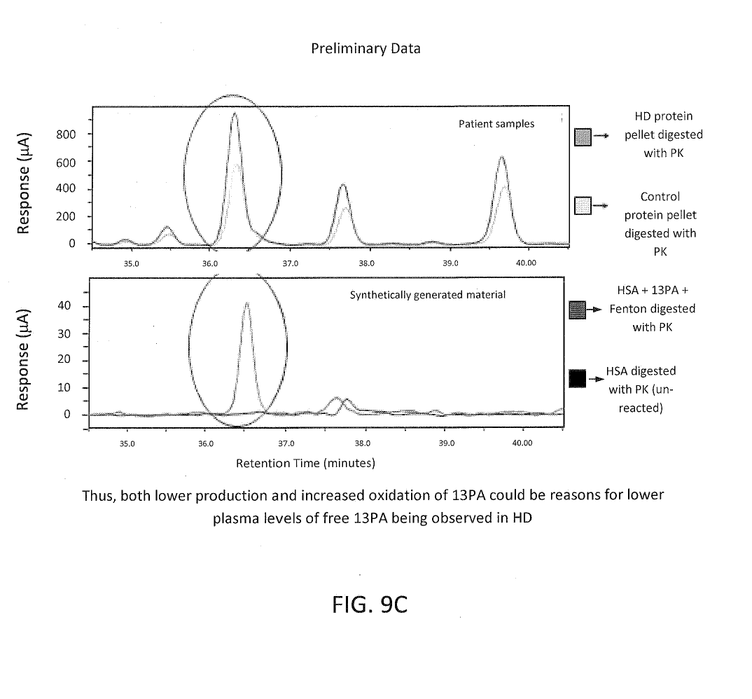

Many of the processes to covalently bind a small molecule to a macromolecule involve the creation of an intermediate small molecule radical by attack with for instance hydroxyl of nitro so radicals. We have made preparations of various proteins, RNA and DNA with coordinate sites saturated with small molecules such as kynurinine or indole propionic acid. These preparations were subjected to free radical attack using various variants of Fenton reactions, peroxide peroxide/nitrate and subsequently processed as above. This allowed the identification of the source of many of the responses as shown in FIGS. 4-8. We used this protocol to identify a species in the PK digests of plasma as a compound formed by free radical attack on indole propionic acid and subsequent binding to protein. This process is illustrated in FIGS. 9A-C. First as shown schematically in FIG. 9A a small molecule was bound to macromolecule (protein or peptide fragments, DNA, RNA etc.) by creating an intermediate free radical of the small molecule in the presence of the macro molecule. In this example we used a traditional Fenton type reaction to create the free radicals. In other applications other means of creating intermediate free radicals such as electrochemical oxidation (i.e. for hydroxy indoles) or UV irradiation (i.e. for non-electrochemically active adducts to DNA or RNA) would be a preferred approach. Second as shown in FIG. 9B the material prepared was concentrated and subjected to Mass Spectrometry to determine the binding site of the amino acid in a protein or peptide and of the base pair in DNA or RNA In this example I3PA was shown to bind as the product of its reactive intermediate kynuric acid to tyrosine. Third synthetic standards were prepared with an appropriate protein for the particular study-in this case evaluating human plasma proteins, human serum albumin (HSA). Coordinately bound materials were extracted identically to the plasma preparations and used to identify the covalently bound species in the human plasma. In this example we identified lower levels of coordinately bound I3PA in controls vs Huntington's Disease subjects consistent with higher levels of oxidative damage in the disease.

Our analysis showed that covalently bound small molecule biomarkers (CBSM) are strong discriminators of state and in the animal data predictors of progression. The distribution of CBSM in protein of other studies lead us to conclude that they also are predictive of outcome of therapy in depression and schizophrenia, time of pheno conversion in Huntington's disease and conversion of mild cognitive impairment to Alzheimer's disease. Thus they are believed to be applicable as biomarkers of state, risk, therapeutic monitoring and prediction in a range of disorders or potentials for disorders.

CBSM also can be used in therapeutic intervention and pharmaceutical development. The rationale for this is that many small molecules are relatively strongly co-ordinately bound to macro molecules. Early risk states whether genetic or induced by environmental factors or by the interaction of genetic and environmental factors as in the case of higher incidence of Amyotrophic Lateral Sclerosis in Gulf War veterans or Parkinson's in agricultural workers exposed to pesticides/herbicides can result in the binding of these small molecules to, for instance, DNA or critical proteins. This binding will in turn affect the operation of the genome (epigenetics) or the functionality of the enzymes. For instance a possibility of the latter effect would be the binding of small molecules to the enzymes in the kynurinine pathway affecting the onset of depression or the outcome of therapy. While not wishing to be bound by theory, it is believed this binding may be the reason that differences in the levels of compounds on this pathway are correlated with depression. Understanding which compounds are bound to the macro molecules provides a route to the design of compounds or strategies to displace them from their coordinate sites with compounds that are not subject to free radical or chemical processes that result in coordinate binding.

Alternatively understanding the compounds covalently associated with DNA allows strategies for design of compounds to specifically change the functionality of the genome-for instance shutting down the function of the breast cancer risk gene by epigenetic covalent binding.

1. A method for determining a disease state of an animal, which comprises determining levels of small molecules covalently bound to macromolecules (CBSM) in samples from that animal, and comparing said levels to standards.

2. The method of claim 1, wherein the CBSM small molecules are sourced from gut microbiome derived, derived from metabolic processes, environmental chemical insult or abnormal chemical environment, endogenous or exogenous microorganism, or an interaction of processes between one or more of the above sources.

3. The method of claim 1, wherein the macromolecule is selected from the group
consisting of DNA, RNA, Protein, a complex carbohydrate and a Glycoprotein.

4. The method of claim 1, wherein the disease state is selected from the group consisting of disease classification, disease sub categorization, disease progression, development of risk factors predictive of disease, specification of therapy, prediction of therapeutic outcome and development of therapeutic leads.

5. The method of claim 1, wherein the animal is a human.

6. A method for therapeutic intervention in disease in an animal comprising manipulating concentration levels of small molecules covalently bound to macromolecules (CBSM).

7. The method of claim 6, wherein the macromolecule is selected from the group
consisting of DNA, RNA, Protein, a complex carbohydrate and a Glycoprotein.

8. The method of claim 6, wherein the small molecules are sourced from gut microbiome derived, derived from metabolic processes, environmental chemical insult or abnormal chemical environment, endogenous or exogenous microorganism, or an interaction of processes between and among one or more the above sources.

9. The method of claim 6, wherein the disease is an affective disease selected from the group consisting of depression, schizophrenia and autism, a degenerative disease selected from the groups consisting of Huntington's, Alzheimer's, Parkinson's, Mild Cognitive impairment, ALS, Freidrich Ataxia, cancer, diabetes, and cardiovascular disease, or from in-born errors of metabolism or genetic based disease.

10. The method of claim 6, wherein the animal is a human.

11. A method of intervention to prevent or ameliorate disease in an animal with disease risk which comprises manipulating the animal's small molecules covalently bound to macromolecules (CBSM).

12. The method of claim 11, wherein the risk is for an affective disease selected from the group consisting of depression, schizophrenia and autism, a degenerative disease selected from the group consisting of Huntington's, Alzheimer's, Parkinson's, Mild Cognitive impairment, ALS or Freidrich Ataxia, cancer, diabetes, and cardiovascular disease or from in-born errors of metabolism or genetic based disease.

13. The method of claim 11, wherein the animal is a human.

14. A method for determining the nature of the source of small molecules covalently bound to macromolecules (CBSM) which comprises creating and analyzing synthetic combinations of small molecules and macromolecules with processes mimicking biochemical processes in an animal.

15. The method of claim 14, wherein the animal comprises a human.

16. A method for modifying gene function in an animal comprising manipulating concentration levels of small molecules covalently hound to macromolecules (CBSM), whereby to increase or decrease expression of a target gene.

17. The method of claim 16, wherein the macromolecule is selected from the group consisting of DNA, RNA, a Protein, a complex carbohydrate and a Glycoprotein.

18. The method of claim 16, wherein the small molecule is sourced from gut microbiome derived, derived from metabolic processes, environmental chemical insult or abnormal chemical environment, endogenous or exogenous microorganism, or an interaction of processes between and among one or more the above sources.

19. The method of claim 16, wherein the gene is associated with an affective disease selected from the group consisting of depression, schizophrenia or autism, a degenerative disease selected from the group consisting of Huntington's, Alzheimer's, Parkinson's, Mild Cognitive impairment, ALS, Freidrich Ataxia, cancer, diabetes, cardiovascular, or from in-born errors of metabolism or genetic based.

20. The method of claim 16, wherein the animal is a human.

21. A method of therapeutic discovery comprising:
identifying a class of subjects with equivalent genetic risk factors;
identifying sub classes in this class who do and do not develop disease;
identifying differences in covalently bound molecules to DNA, RNA and protein that discriminate the class and subclasses and affect epigenetic differences in the system feedback control;
isolating and determining chemical precursor sources and structures of the covalently bound discriminators; and
providing or replacing such compounds as are missing or in reduced amount in the disease developing class and/or suppressing such compounds that are elevated or in excess in the disease developing class.

22. The method of discovery in 21 wherein the disease is a neurodegenerative disease selected from the group consisting of Huntington's, Parkinson's, Mild Cognitive Impairment, Amyotrophic Lateral Sclerosis, Freidrich Ataxia, cancer, diabetes and cardiovascular disease, an affective disorder selected from the group consisting of depression, schizophrenia and autism, or from in-born errors of metabolism or genetic based disease.

23. Small molecules covalently bound to macromolecules for determination of disease risk, diagnostic status, prediction of progression and development of therapy.
